(19) United States
(12) Patent Application Publication
(10) Pub. No.: US 2009/0204039 A1
(43) Pub. Date: Aug. 13, 2009

(54) Device and Method for Treating Neck Tension or Neck Injury

(75) Inventors: Ori Elan, Tel-Aviv (IL); Yoav Tikochinsky, Tel-Aviv (IL)

Correspondence Address: MARTIN D. MOYNIHAN d/b/a PRTSI, INC., P.O. BOX 16446, ARLINGTON, VA, 22215, US

(73) Assignee: BACKLIFE LTD., Or Yehuda (IL)

(21) Appl. No.: 11/922,122

(22) PCT Filed: Mar. 13, 2006
(86) PCT No.: PCT/IL06/00327

§ 371 Date: Dec. 29, 2008

Related U.S. Application Data
(60) Provisional application No. 60/661,019, filed on Mar. 14, 2005.

(51) Int. Cl. A61F 5/00

(52) U.S. Cl. 602/18; 606/237

(57) ABSTRACT

A device for treating neck tension or neck injury is provided. The device includes a base connected to a head support which is capable of continuous bi-directional movement relative to at least one axis of said base. The device is configured such that the continuous bi-directional movement is capable of repetitively flexuring and/or extending neck vertebrae of a user of the device.

### FIELD AND BACKGROUND OF THE INVENTION

The present invention relates to a device for treating neck tension or neck injury and, more particularly, to a continuous passive motion (CPM) device which is capable of repetitively flexuring and/or extending neck vertebrae of a user.

Joint and muscle injury whether due to trauma (e.g., sports injury) or repetitive stress (e.g., repetitive stress injury—RSI) afflicts most of the western population and is one of the main reasons for loss of productivity at the work place.

Although numerous therapeutic devices and methods have been designed for the sole purpose of preventing or treating joint and muscle injury, early devices were primarily designed for joint/muscle immobilization.

In the last few decades practitioners have realized that immobilization is unhealthy for joints since it can create deleterious sequelae of physiological and functional impairments. To traverse the limitations of immobilization therapy, Robert B. Salter, MD, a Toronto orthopedic surgeon, first pioneered the biological concept of continuous passive motion (CPM) more than 30 years ago, as a means of healing and regenerating joint cartilage. The fact that patients could not be expected to move their own injured joints continuously for hours at a time, led to the development of CPM devices.

Through the years, CPM devices have been created for most of the major joints of the upper and lower extremities, but they appear to be most frequently prescribed for use in postsurgical injuries of the knee or shoulder. They are generally provided through devices that are mechanically designed to bend and flex joints at a given rate over a period of several hours.

Examples of CPM devices include the Phoenix Model series knee CPM devices and the MTL 2470 Shoulder CPM device manufactured by Mckelor Technologies Ltd., and the Optiflex line of CPM devices manufactured by Lifetec Inc.

Research conducted with CPM devices has shown that CPM mediated treatment can deliver orthopedic, neurological, and even circulatory benefits to the patient.

The neck (cervical spine) is composed of vertebrae which begin in the upper torso and end at the base of the skull. The bony vertebrae along with the ligaments provide stability to the spine. The muscles allow for support and motion. The neck has a significant amount of motion and supports the weight of the head. However, because it is less protected than the rest of the spine, the neck can be vulnerable to injury and disorders that produce pain and restrict motion.

Neck pain may result from abnormalities in the soft tissues—the muscles, ligaments, and nerves—as well as in bones and joints of the spine. The most common causes of neck pain are soft tissue abnormalities due to injury or prolonged wear and tear. One example of a common neck injury is whiplash injury which is clinically defined as hyperextension/hyperflexion injury of the neck. It is estimated that in the United States alone there are one million whiplash injuries from car accidents a year (http://www.whiplash-car-accidents.com/pages/whiplash.html). Although the exact number of whiplash injuries varies from one country to another, it is estimated that the incidence of whiplash injury is approximately 1 in 1000 people in western society.

Neck injuries are typically treated via physical therapy and massaging/traction devices. Typical devices include neck wraps (see for example, the Neck Pain Wrap, available through the beanbagsuperstore), massaging devices (see for example, www.interhealth.com/consumer/neck/cms300/index.htm), or traction devices such as the Neckpro™ Overdoor Cervical Traction Device or the comfortrac™ home cervical traction. Additional examples of neck treatment devices includes the device described in U.S. Pat. No. 6,159,169 which is designed for treating whiplash injury by rotating the users head from side-to-side thus exercising neck muscles and the chair-mounted device described in U.S. Pat. No. 5,116,359 which is designed for exercising the head neck and shoulder muscles. U.S. Pat. No. 5,192,306 describes a chiropractic manipulation table with flexion/distraction headpiece. Although this device is capable of inducing continuous passive neck movement, it requires a chiropractic operator and is thus incapable of autonomous distraction/flexion operation cycles.

Although the above described devices offer some degree of relief for neck pain, their therapeutic value is limited since they are incapable of inducing continuous passive movement especially of the neck vertebrae.

While reducing the present invention to practice the present inventors have applied knowledge gained from developing the back CPM device, BackLife™, to a neck specific CPM device which is capable of repetitively flexuring, extending or distracting-flexuring neck vertebrae of a user and as such enable effective treatment of a wide range of neck injuries.

### SUMMARY OF THE INVENTION

According to one aspect of the present invention there is provided a device for treating neck tension and/or neck injury comprising a base connected to a head support, the head support being capable of continuous bi-directional movement relative to at least one axis of the base, wherein the device is configured such that the continuous bi-directional movement is capable of inducing angular cervical intervertebral motion thereby repetitively flexuring and/or extending neck vertebrae of a user of the device.

According to another aspect of the present invention there is provided a method of treating neck tension and/or neck injury of an individual comprising: (a) placing a head of the individual in a head support capable of continuous bi-directional movement relative to at least one axis of a base connected thereto; (b) activating the continuous bi-directional movement to thereby repetitively flex and/or extend neck vertebrae and treat neck tension or neck injury of the individual.

According to further features in preferred embodiments of the invention described below, the continuous bi-directional movement is also capable of inducing translational motion of the vertebral centroid of the neck vertebrae.

According to still further features in the described preferred embodiments a cycle of the continuous bi-directional movement induces the translational motion of the vertebral centroid of the neck vertebrae followed by the angular cervical intervertebral motion.

According to still further features in the described preferred embodiments the continuous bi-directional movement follows a substantially planar path.

According to still further features in the described preferred embodiments the head support includes a thermal element for cooling or heating a neck region of the user.

According to still further features in the described preferred embodiments the continuous bi-directional movement follows an elliptical path.

According to still further features in the described preferred embodiments the head support is adapted to support an occiput region of the user.

According to still further features in the described preferred embodiments the head support is configured such that force is applied on the neck vertebrae in one direction of the bi-directional movement.

According to still further features in the described preferred embodiments the base is adapted for positioning on a floor surface.

According to still further features in the described preferred embodiments the base is adapted for placement on a body region of the user.

According to still further features in the described preferred embodiments the device further comprises a power unit for generating the continuous bi-directional movement of the head support.

According to still further features in the described preferred embodiments the power unit includes an electric motor or servo.

According to still further features in the described preferred embodiments the device further comprises a user interface for controlling operation of the device.

According to still further features in the described preferred embodiments the device further comprises a power source for powering the power unit.

According to still further features in the described preferred embodiments a transition from a first direction to a second direction of the continuous bi-directional movement of the head support of the device is effected autonomously.

According to still further features in the described preferred embodiments the continuous bi-directional movement of the head support of the device is autonomous.

According to still further features in the described preferred embodiments the user interface enables the user to set a predetermined time of operation.

According to still further features in the described preferred embodiments the continuous bi-directional movement of the head support is effected via autonomous inflation/deflation of a bladder.

According to still further features in the described preferred embodiments the bladder forms a part of the head support or the base.

The present invention successfully addresses the shortcomings of the presently known configurations by providing a device capable of subjecting neck vertebrae and/or neck and shoulder muscles of a user to continuous cyclic movement thereby greatly facilitating treatment of vertebra and/or muscle related neck and shoulder disorders.

Unless otherwise defined, all technical and scientific terms used herein have the same meaning as commonly understood by one of ordinary skill in the art to which this invention belongs.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are schematic diagrams illustrating extension (FIG. 1a) and flexion (FIG. 1b) of neck vertebrae, insets illustrate the effect of extension or flexion on inter-vertebrae space;

FIG. 2 is a perspective view of one embodiment of the device of the present invention;

FIG. 3 illustrates a device constructed according to the teachings of the present invention and having a head support capable of moving through a linear/planar path;

FIG. 4 illustrates a device constructed according to the teachings of the present invention and having a head support capable of moving through an arced path;

FIG. 5 illustrates a device constructed according to the teachings of the present invention and having a head support capable of moving through a linear-arced path;

FIGS. 6a-b illustrate a device constructed according to the teachings of the present invention in which the head support is moved through a path via a powered pulley/strap mechanism; and

FIG. 7 is an image of a prototype of the device of the present invention.

### DESCRIPTION OF THE PREFERRED EMBODIMENTS

The present invention is of a neck CPM device which can be used to treat neck tension or neck injury. In particular, the CPM device of the present invention is designed capable of subjecting neck vertebrae of a user to continuous (cyclic) cycles of vertebrae flexion/extension and/or distraction.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Treatment of neck injuries and disorders is typically effected using various traction and/or massaging devices or via physical therapy. Although numerous CPM devices have been described in the prior art, none are specifically designed for and thus are incapable of, repetitively flexuring and/or extending neck vertebrae.

The vast knowledge gained in designing and constructing back CPM devices has enabled the present inventors to devise and construct a neck CPM device which is capable of flexuring and/or extending cervical vertebrae of a user without the need for user or practitioner intervention.

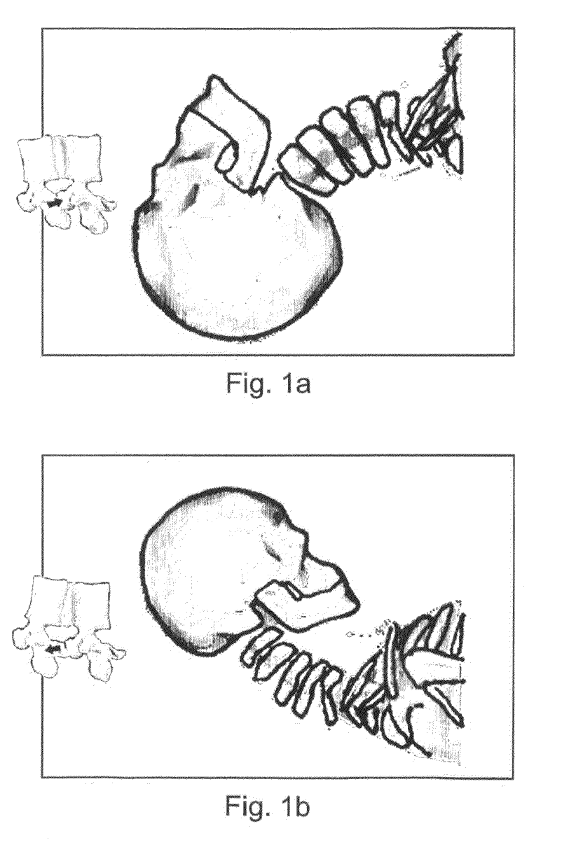

As used herein, the terms “flexion” and “flexuring” when used in reference to the neck, refer to the act of inducing angular cervical intervertebral motion via anterior head movement (chin in). As used herein, the terms “extension” when used in reference to the neck, refers to the act of inducing angular cervical intervertebral motion via posterior head movement (chin up). Both flexion and extension result in angular cervical intervertebral separation; extension and flexion are illustrated in FIGS. 1a-b respectively. The term “distraction” is used herein to denote a symmetric pulling force on neck vertebrae; distraction results in symmetric cervical intervertebral separation.

As used herein, the term “continuous” when used with respect to the movement of the head support of the device of the present invention refers to movement through a predetermined path which is controlled by the device without need for user or operator intervention. Such movement can be of varying velocity and acceleration, including points in the path where velocity equals zero (e.g., points in the path where head support movement is stopped by the device for predetermined time periods, to enable, for example, temporary traction). Thus, the term continuous refers to the ability of the device of the present invention to continuously cycle movement of the head support through the path and not to the velocity of the head support when moving through the path.

According to one aspect of the present invention there is provided a device for treating neck tension and/or neck injury which is caused by RSI, trauma or disease. It will be appreciated that although the present device is specifically targeted at relief and/or treatment of neck injuries, it could also be utilized to treat shoulder muscle tension since such tension often originates from neck injuries or tension or stress on neck vertebrae.

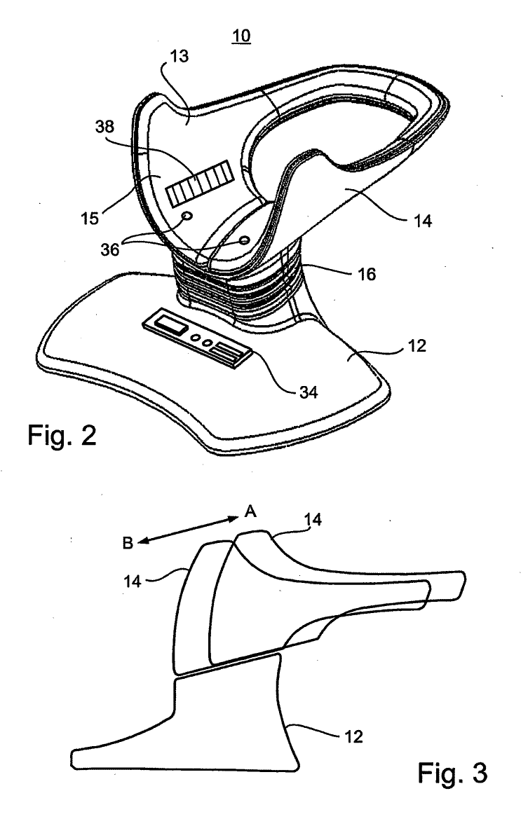

As is described hereinunder and illustrated in FIGS. 2-7, the device of the present invention is configured capable of repetitively flexuring and/or extending neck vertebrae of a user (e.g., human subject) thereof.

FIGS. 2-7 illustrate various embodiments of the device of the present invention, which is referred to herein as device 10. Device 10 can be constructed from polymeric material or a combination of metals and polymeric material. The various device body components described below are preferably fabricated as molded polymeric shells fitted over a metallic, polymeric or wood substructure. Based on the descriptions and Figures provided herein, one of ordinary skill in the art would be readily capable of selecting the appropriate materials and fabrication processes suitable for fabricating device 10 of the present invention.

As is illustrated in FIG. 2, device 10 includes a base 12 which is connected to a head support 14. Base 12 is configured for placement on a floor, treatment table or a body region of the user (e.g., shoulders). FIGS. 2-7 illustrate a floor/treatment table supported base 12 configuration. Head support 14 is adapted to support the back of the user's head (at 13), preferably an occiput region thereof. Head support 14 can include straps for securing the head, although head support 14 having an occiput supporting configuration (e.g., a support having variable width) can forgo the use of straps. Since device 10 can be in operation for several minutes or more, support surface 15 of head support 14 also includes cushioning material, such as foam, or an elastomeric material.

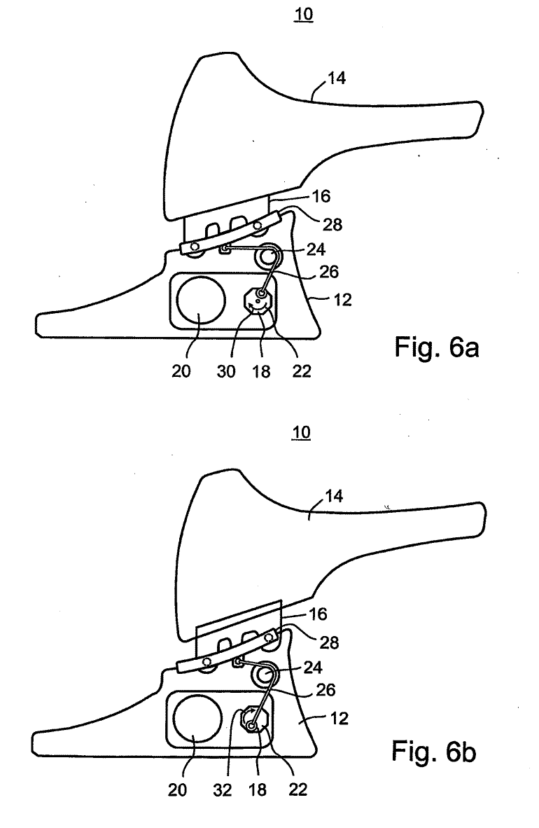

Connection between base 12 and head support 14 can be effected via a connecting element 16 which can be a link or strut, although other connection means such as cables or inflatable bladders are also envisaged (see FIGS. 6a-b for example). Connecting element 16 and its attachment to base 12 and head support 14 is configured so as to allow head support 14 to move continuously in a bi-directional path relative to at least one axis (X, Y and/or Z) of base 12.

Device 10 is configured such that the continuous bi-directional movement of head support 14 (see FIGS. 3-5, superimposed images of head support 14 illustrates extent of movement) results in repetitively flexuring and/or extending or preferably repetitively flexuring/distracting neck vertebrae of a user of the device.

To enable distraction, head support 14 can follow a bidirectional planar path along the X-axis of base 12; in this case, head support 14 repetitively (symmetrically) separates neck vertebrae of a user of device 10, since head support 14 moves away from base 12 (see FIG. 3 arrow A) in a planar path and returns in a reverse direction along same path (arrow B).

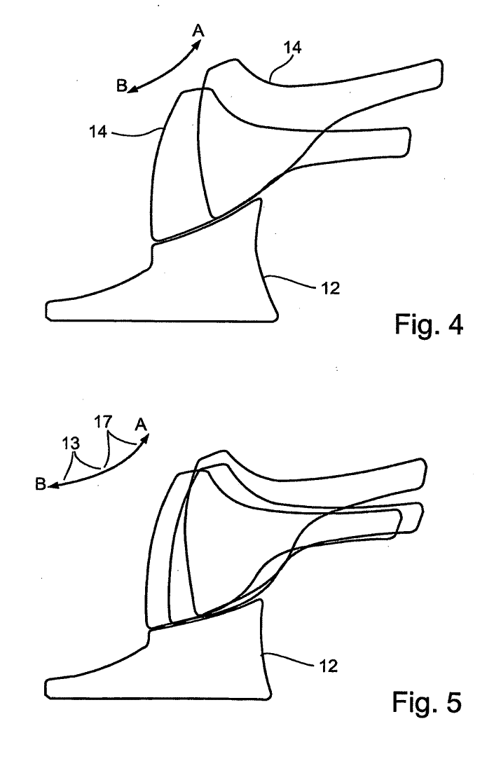

To enable flexuring, head support 14 can follow an elliptical or bow-shaped path along the X-axis of base 12. When moving away from base 12 (indicated by arrow A in FIG. 4), head support follows an arced path which rises as head support 14 moves away from base 12 (along the X-axis). On a return path, head support 14 can follow the path described above (in a reverse direction, arrow B) or it can follow a planar path. The end effect of such movement is titling of a head (chin in) of a user, effectively flexuring cervical vertebrae. This movement essentially mimics manipulations conducted by a physical therapist when treating neck tension or injury. It will be appreciated that repetitive extension (chin up) cycles can be achieved by inverting the path described above.

To enable flexuring-distraction, head support 14 can follow a planar-elliptical or bow-shaped path along the X-axis of base 12. When moving away from base 12 (arrow A in FIG. 5), head support can follow a path which is composed of a planar component 13 followed by an arced component 17 which rises as head support 14 moves away from base 12 (along the X-axis). On a return path, head support 14 can follow the path described above (in a reverse direction, arrow B) or a planar path. The end effect of such movement is both distraction and titling of a head of a user, effectively distracting and flexuring cervical vertebrae. This movement essentially mimics manipulations conducted by a physical therapist when treating neck tension or injury.

Head support 14 can follow additional paths when device 10 is in operation. For example, in addition to the paths described above, head support 14 can also rotate from side to side while in operation thus rotating a user's head from side to side while applying extension or flexuring forces. Alternatively, head support 14 can also tilt side to side (ear to shoulder).

Although movement of head support 14 of device 10 can be generated by a user or operator of device 10 (for example, via hand operation of a crank), device 10 is preferably capable of autonomous operation which can be induced by commands of a user or operator.

To enable autonomous movement of head support 14, device 10 includes a power unit 18 and a power supply 20 (FIGS. 6a-b). Examples of suitable power units include an electric motor which can serve as a drive or a pump (further described below), an electric servo, or the like.

An electric motor is preferred for its silent operation, long life and compactness. Power supply 20 for an electric motor can be a DC current provided by, for example, a battery or an AC current, provided by, for example, a wall socket.

When utilized as a drive, an electric motor can be connected to connecting element 16 via gears, cams and/or pulleys; alternatively, the gears, cams and/or pulleys can for a part of connecting element 16.

Torque supplied by the electric motor can be converted to repetitive bidirectional movement via any one of several gear/pulley/cam arrangements. FIGS. 6a-b illustrates one such arrangement in which electric motor 22 (which in this embodiment is sequestered within base 12) drives head support 12 via a pulley 24 and strap 26. As is shown in FIGS. 6a-b, rotation of electric motor 22 (arrow 30, FIG. 6a) pulls on strap 26 (guided by pulley 24) which in turn pulls head support 14 through a path set by guide 28. Further rotation of electric motor 22 (arrow 32, FIG. 6b) releases strap 26 and returns head support 14 to the position illustrated in FIG. 6a.

The electric motor utilized by device 10 of the present invention can alternatively function as a pump which can repetitively inflate and deflate a bladder serving as connecting element 16. With such a design, inflation of the bladder effectively moves head support 14 along a path (which follows an axis of base 12) while deflation of the bladder moves the head support in return path. Such a bladder can be constructed from an resilient airtight polymeric material such as Silicone.

Device 10 of the present invention preferably also includes a user interface 34 (shown in FIG. 2) which enables a user or operator to switch on device 10 and to set user-specific treatment parameters such as length of treatment, degree of extension/flexion, path of movement of head support 14, and to set user-specific parameters such as height angle and support surface area of head support 14 (to accommodate for variance in neck length head size etc).

User interface 34 can also include, or be in communication with a microprocessor for controlling operation of device 10, for inputting user specific settings (e.g., age, gender, height weight, type of pain etc) and for optionally collecting data on user treatment regimens, issuing warnings when regimen exceeds optimal range of motion, time and the like.

In order to adjust treatment according to user preferences or disorder, device 10 enables setting the velocity (and/or acceleration) of head support 14 movement and the extent of distraction or flexion/extension applied to the user's neck vertebrae. Such setting can be achieved by varying the range of motion and/or velocity/acceleration of head support 14 relative to base 12 or the angle at which head support rises when following the arced path described above or by setting the number of cycles completed per minute. Parameters which relate to such settings can be set by a user or operator via user interface 34 or adjusted automatically by device 10 when in operation. The latter can be achieved by providing head support 14 with force or load sensors 36 (such as those available from Tekscan or MSI sensors) which can determine the force applied to the head of the user (e.g., the occiput region) when device 10 is in operation, or by measuring the load on, for example, electric motor 22. Such load or pressure data can be processed by microprocessor of device 10 and converted into commands for adjusting angle (of path), acceleration and/or velocity accordingly.

Head support 14 can also include sensors for measuring a physiological parameter associated with neck muscle or vertebrae.

For example, surface myography sensors, such as those available from Thought Technology Ltd. (http://www.thoughttechnology.com/sensors.htm) or Schuhfried (www.schuhfried.co.at/eng/biofeedback/physiorecorder_mehr_info.htm) can be integrated into, or attached to, head support 14 and utilized to measure neck muscle stiffness and/or to assess muscle healing and reconditioning. Data collected by such sensors can be processed (through a microprocessor controller of device 10) and used to adjust (preferably automatically) the operation of device 10, prior to, or during treatment.

Other sensors including, for example, blood flow and temperature sensors can also be integrated into, or attached to, head support 14 of device 10 and used in a similar manner.

Additional description with respect to device 10 setting and treatment regimens is provided hereinbelow.

Head support 14 can also include additional features which may assist in treatment or increase the comfort level of a user of device 10.

For example, head support 14 can include thermal elements 38 (one shown in FIG. 2) which can be used to heat or cool the neck region of the user. Such a thermal element can be a heating element, a Peltier element, a combination thereof or the like. Heating a neck region of the user can increase blood flow through the area and combined with extension/flexion can increase the healing efficacy of the device by improving muscle and cartilage healing via increased nourishment and more efficient removal of waste products such as lactic acid.

Since device 10 can be in use over extended time periods, head support 14 can also include an entertainment device such as, for example, a music player (e.g., MP3 player, radio) having speakers or headphone jack integrated into the head rest; transfer of music files to such a player can be facilitated via a removable memory card (e.g., Flash memory card).

As is mentioned hereinabove, device 10 of the present invention can be utilized to treat (e.g., relieve) neck tension and/or treat neck injuries of a user.

Treatment regimen is set according to the severity of tension or injury. Typically, the user or operator of device 10 will set a timed session of movement and extent of movement according to physician advice or personal experience. Guidelines which can be utilized for determining range of motion of head support 14 can be found in physiotherapy treatment protocols (see, for example, “Steps to a Pain-Free Life: How to Rapidly Relieve Back and Neck Pain” by Robin McKenzie and Craig Kubey, Plume Books; Reissue edition; Oct. 1, 2001 or “The American Physical Therapy Association Book of Body Maintenance and Repair” by Marilyn Moffat, Terry Boles, Steve Vickery, American Physical Therapy Association; Henry Holt & Company; Apr. 1, 1999). A typical range of motion (of head support 14) which can be utilized for treatment of neck tension/injury is 1-20 cm, while a typical pulling/bending force applied to the neck region can be in a range equivalent to the force generated by about 0.5-5 Kg of weight.

Device 10 of the present invention can be utilized to treat a variety of neck injuries. For example, neck/shoulder stress caused by repeated/prolonged use of, for example, a personal computer can lead to muscle spasms, pain, local inflammation and limiting of cervical motion. Typical treatment of such injuries includes: (i) instructions for correct posture; (ii) exercises for the neck and shoulder girdle; (iii) local heating; (iv) mobilization of the neck vertebrae; (v) massaging/movement of neck muscles; and(vi) traction.

Device 10 of the present invention can be used to provide (i), (ii) and (v) and (vi) by enabling exercising of the neck region through continuous flexion/extension cycles (4-15 cycles per minute, at a force equivalent to about 0.5-5 Kg), including provision of temporary traction (e.g., by suspending head support movement for a predetermined time period) while optionally providing neck rotation and side to side motion. Device 10 can also provide heating to the neck region for stimulating muscle and cartilage repair and temporary or intermittent traction when necessary.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLE

Reference is now made to the following example, which together with the above descriptions, illustrate the invention in a non limiting fashion.

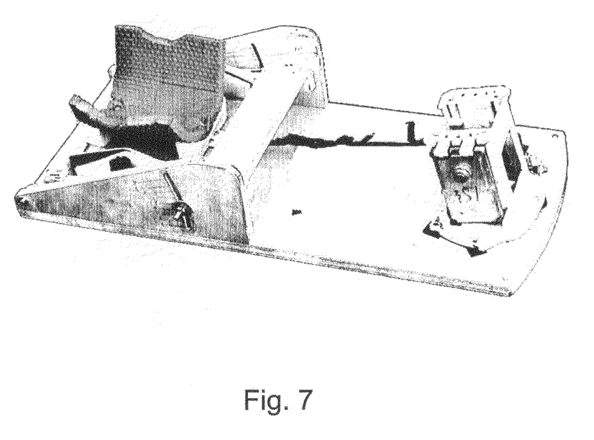

A prototype of the CPM neck device of the present invention (FIG. 7) was presented for evaluation to a panel of 12 of the leading medical practitioners in the field of orthopedics and orthopedic surgery in Israel. Each panel member was subjected to a 10-minute treatment session using this prototype. Provided below is a summary of their preliminary conclusions.

The panel members unanimously concluded that:
1. the device mobilizes the neck aria and provides a range of motion which is similar to that applied by neck physiotherapists and that such movement can be effective in treatment and alleviation of neck pain;
2. there is a minimal risk factor if the motion is limited to the mid range as specified in physiotherapy treatment protocols;
3. in cases were there is no fraction or neurological damage—candidates for physiotherapy can be treated by the device of the present invention with no risk; and
4. there is an unmet need for a device that applies CPM to neck vertebrae.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A device for treating neck tension and/or neck injury comprising a base connected to a head support, said head support being capable of continuous bi-directional movement relative to at least one axis of said base, wherein the device is configured such that said continuous bi-directional movement is capable of inducing angular cervical intervertebral motion thereby repetitively flexuring and/or extending neck vertebrae of a user of the device.

2. The device of claim 1, wherein said continuous bi-directional movement is also capable of inducing translational motion of the vertebral centroid of said neck vertebrae.

3. The device of claim 2, wherein a cycle of said continuous bi-directional movement induces said translational motion of the vertebral centroid of said neck vertebrae followed by said angular cervical intervertebral motion.

4. The device of claim 1, wherein said continuous bi-directional movement follows a substantially planar path.

5. The device of claim 1, wherein said head support includes a thermal element for cooling or heating a neck region of the user.

6. The device of claim 1, wherein said continuous bi-directional movement follows an elliptical path.

7. The device of claim 1, wherein said head support is adapted to support an occiput region of said user.

8. The device of claim 1, wherein said head support is configured such that force is applied on said neck vertebrae in one direction of said bi-directional movement.

9. The device of claim 1, wherein said base is adapted for positioning on a floor surface.

10. The device of claim 1, wherein said base is adapted for placement on a body region of said user.

11. The device of claim 1, further comprising a power unit for generating said continuous bi-directional movement of said head support.

12. The device of claim 11, wherein said power unit includes an electric motor or servo.

13. The device of claim 1, further comprising a user interface for controlling operation of the device.

14. The device of claim 11, further comprising a power source for powering said power unit.

15. The device of claim 1, wherein a transition from a first direction to a second direction of said continuous bi-directional movement of said head support of the device is effected autonomously.

16. The device of claim 11, wherein said continuous bi-directional movement of said head support of the device is autonomous.

17. The device of claim 13, wherein said user interface enables the user to set a predetermined time of operation.

18. The device of claim 1, wherein said continuous bi-directional movement of said head support is effected via autonomous inflation/deflation of a bladder.

19. The device of claim 18, wherein said bladder forms a part of said head support or said base.

20. A method of treating neck tension and/or neck injury of an individual comprising:
(a) placing a head of the individual in a head support capable of continuous bi-directional movement relative to at least one axis of a base connected thereto;
(b) activating said continuous bi-directional movement to thereby repetitively flex and/or extend neck vertebrae and treat neck tension or neck injury of the individual.

21. The method of claim 20, wherein said continuous bi-directional movement follows a substantially planar path.

22. The method of claim 20, wherein said head support includes a thermal element for cooling or heating a neck region of the individual.

23. The method of claim 20, wherein said continuous bi-directional movement follows an elliptical path.

24. The method of claim 20, wherein said head support is adapted to support an occiput region of the individual.

25. The method of claim 20, wherein said head support is configured such that force is applied on said neck vertebrae in one direction of said bi-directional movement.

26. The method of claim 20, wherein said base is adapted for positioning on a floor surface.

27. The method of claim 20, wherein said base is adapted for placement on a body region of said user.

28. The method of claim 20, wherein said continuous bi-directional movement is generated by a power unit.

29. The method of claim 28, wherein said power unit includes an electric motor or servo.

30. The method of claim 20, wherein a transition from a first direction to a second direction of said continuous bi-directional movement of said head support is effected autonomously.

31. The method of claim 20, wherein said continuous bi-directional movement of said head support of the device is autonomous.

32. The method of claim 20, wherein said continuous bi-directional movement of said head support is effected via autonomous inflation/deflation of a bladder.

33. The method of claim 32, wherein said bladder forms a part of said head support or said base.
